(19) United States
(12) Patent Application Publication
(10) Pub. No.: US 2023/0398059 A1
(43) Pub. Date: Dec. 14, 2023

(54) OIL-BASED COSMETIC

(71) Applicant: SHISEIDO COMPANY, LTD., Chuo-ku, Tokyo (JP)

(72) Inventors: Kyohei KOSAKA, Tokyo (JP); Kiriko CHIBA, Tokyo (JP)

(73) Assignee: SHISEIDO COMPANY, LTD., Chuo-ku, Tokyo (JP)

(21) Appl. No.: 18/033,144

(22) PCT Filed: Oct. 18, 2021
(86) PCT No.: PCT/JP2021/038455

§ 371 Date: Apr. 21, 2023

(30) Foreign Application Priority Data
Oct. 30, 2020 (JP) 2020-182803

(51) Int. Cl. A61K 8/92; A61Q 1/06

(52) U.S. Cl. CPC A61K 8/92; A61Q 1/06; A61K 2800/91; A61K 2800/434; A61K 2800/48

(57) ABSTRACT

To provided provide an oil-based cosmetic in which a pattern such as uneven or streak-like flow marks is reduced or inhibited, and which has excellent appearance gloss.

An oil-based cosmetic of the present disclosure contains a first photoluminescent pigment having an average particle size of 80 μm or less; and a polymeric oil-phase thickener, wherein the mass ratio of the first photoluminescent pigment and a second photoluminescent pigment having an average particle size of more than 80 μm is 55:45 to 100:0, when the oil-based cosmetic contains a coloring material, the mass ratio of the first photoluminescent pigment with respect to the coloring material is 2.0 or higher, the content of thickening particles is less than 4.0% by mass, and the oil-based cosmetic has a viscosity of 50 mPa·s or higher under a temperature in at least a portion of a range of 80 to 150° C.

### FIELD

The present disclosure relates to an oil-based cosmetic.

### BACKGROUND

A variety of oil-based cosmetics have been developed in recent years.

PTL 1 discloses stick-shaped oil-based solid cosmetics having a transparent outer layer part and a colored inner layer part that are in contact with each other, the outer layer part containing at least a component (A): dibutyl lauroyl glutamide, a component (B): dibutyl ethylhexanoyl glutamide, and a component (C): a polyglycerol fatty acid ester in which the polymerization degree of glycerol is 6 to 12 and the fatty acid is a branched fatty acid having 8 to 22 carbon atoms.

PTL 2 discloses a stick-like cosmetic which contains (a) a polyethylene wax having a crystallization starting temperature of 80 to 95° C. and (b) a microcrystalline wax having a crystallization starting temperature of 85 to 100° C. at a mass ratio (a):(b) of 99:1 to 60:40, and further contains (c) a liquid oil component having an IOB value of 0.2 or more in an amount of 25 not less than 30% by mass.

### CITATION LIST

### Patent Literature

[PTL 1] Japanese Unexamined Patent Publication (Kokai) No. 2017-119698

[PTL 2] Japanese Unexamined Patent Publication (Kokai) No. 2008-133205

### Technical Problem

### SUMMARY

There are known technologies for imparting the lips with a glitter texture such as a pearly texture by, for example, incorporating a colored photoluminescent pigment or a combination of a colorless or white photoluminescent pigment and a coloring material into an oil-based cosmetic such as a lipstick.

Oil-based cosmetics such as lipsticks are prepared by, for example, heat-melting a solid preparation such as a wax, mixing the thus molten preparation with additives such as a pigment, injecting the resulting mixture into a mold, and then cooling the mixture. In this process, the use of, for example, a colored photoluminescent pigment or a combination of a colorless or white photoluminescent pigment and a coloring material as an additive sometimes leads to the formation of a pattern such as the uneven or streak-like flow marks depicted in FIG. 1(a). Even if an oil-based cosmetic having such a pattern is used as, for example, a lipstick and applied to the lips, a color tone and a glitter texture are sufficiently obtained as depicted in FIG. 1(b); therefore, conventionally, oil-based cosmetics are often commercialized in the state of having a pattern such as uneven or streak-like flow marks in appearance.

However, in recent years, greater importance is increasingly attached to the appearance aesthetics of oil-based cosmetics themselves such as lipsticks, in addition to the performance such as simple color retention and good color development.

Therefore, a main object of the present disclosure is to provide an oil-based cosmetic in which a pattern such as uneven or streak-like flow marks is reduced or inhibited, and which has excellent appearance gloss.

### Solution to Problem

<Aspect 1>

An oil-based cosmetic, containing: a first photoluminescent pigment having an average particle size of 80 μm or less; and a polymeric oil-phase thickener,
wherein
the mass ratio of the first photoluminescent pigment and a second photoluminescent pigment having an average particle size of more than 80 μm is 55:45 to 100:0,
when the oil-based cosmetic contains a coloring material, the mass ratio of the first photoluminescent pigment with respect to the coloring material is 2.0 or higher, the content of thickening particles is less than 4.0% by mass, and
the oil-based cosmetic has a viscosity of 50 mPa·s or higher under a temperature in at least a portion of a range of 80 to 150° C.

<Aspect 2>

The cosmetic according to aspect 1, wherein the content of the first photoluminescent pigment is 2.0% by mass or more.

<Aspect 3>

The cosmetic according to aspect 1 or 2, wherein the polymeric oil-phase thickener contains a copolymer having at least one selected from an ethylene monomer unit and a styrene monomer unit.

<Aspect 4>

The cosmetic according to any one of aspects 1 to 3, further containing a hydrocarbon oil.

<Aspect 5>

The cosmetic according to any one of aspects 1 to 4, containing a coloring material,

wherein the first photoluminescent pigment is a colorless or white photoluminescent pigment.

<Aspect 6>

The cosmetic according to any one of aspects 1 to 5, which is used as a lipstick.

<Aspect 7>

A method of producing the cosmetic according to any one of aspects 1 to 6, the method comprising:

melting a raw material mixture, which contains the first photoluminescent pigment and the polymeric oil-phase thickener, at a temperature of 80 to 150° C. at which the mixture has a viscosity of 50 mPa·s or higher; and
injecting the thus molten mixture into a mold.

### Advantageous Effects of Invention

According to the present disclosure, an oil-based cosmetic, in which a pattern such as uneven or streak-like flow marks (hereinafter, may be simply referred to as “flow marks”) is reduced or inhibited, and which has excellent appearance gloss, can be provided.

### BRIEF DESCRIPTION OF DRAWINGS

FIG. 1(a) is an appearance photograph of a stick-like oil-based cosmetic having flow marks, and FIG. 1(b) is an appearance photograph that was taken after applying the cosmetic to the skin.

FIG. 2(a) is an appearance photograph of a stick-like oil-based cosmetic which is reduced in flow marks but has poor surface gloss in appearance, and FIG. 2(b) is an appearance photograph of a stick-like oil-based cosmetic according to one embodiment of the present disclosure which is reduced in flow marks and has excellent surface gloss in appearance.

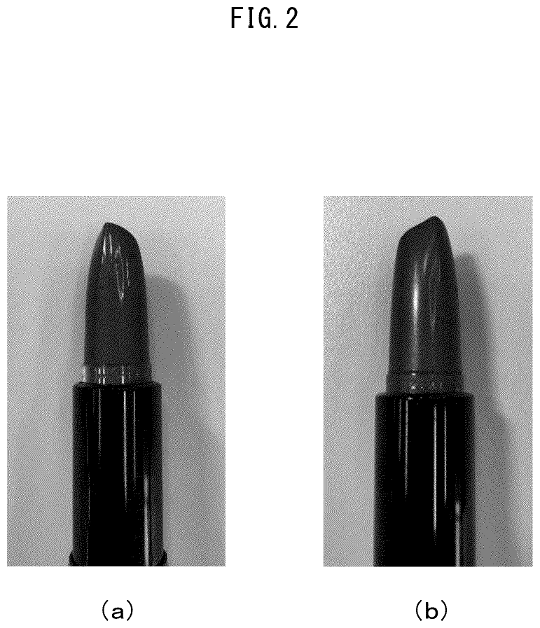

### DESCRIPTION OF EMBODIMENTS

Embodiments of the present disclosure will now be described in detail. The present disclosure is, however, not limited to the below-described embodiments, and can be carried out with various modifications within the gist of the invention.

The oil-based cosmetic (hereinafter, may be simply referred to as “cosmetic”) of the present disclosure contains: a first photoluminescent pigment having an average particle size of 80 μm or less; and a polymeric oil-phase thickener, and is configured such that: the mass ratio of the first photoluminescent pigment and a second photoluminescent pigment having an average particle size of more than 80 μm is 55:45 to 100:0; when the oil-based cosmetic contains a coloring material, the mass ratio of the first photoluminescent pigment with respect to the coloring material is 2.0 or higher; the content of thickening particles is less than 4.0% by mass; and the oil-based cosmetic has a viscosity of 50 mPa·s or higher under a temperature in at least a portion of a range of 80 to 150° C.

Without being bound by any principle, the action principle in which a pattern such as uneven or streak-like flow marks is reduced or inhibited and excellent appearance gloss is attained in such an oil-based cosmetic is believed to be as follows.

For example, a stick-like oil-based cosmetic is generally prepared by injecting a mixture, which contains a heat-melted solid preparation such as a wax and additives such as a photoluminescent pigment, into a mold from above, and subsequently cooling the mixture. It is believed that, in this process, since convection associated with the injection occurs inside the mold, and the photoluminescent pigment and the like are oriented along the flow of the convection and solidified by the cooling in this state, uneven or streak-like flow marks appear in the appearance of the resulting stick. The oil-based cosmetic of the present disclosure is designed such that the use of a polymeric oil-phase thickener therein allows the oil-based cosmetic to exhibit a viscosity of 50 mPa·s or higher under a temperature in at least a portion of a range of 80 to 150° C., for example, under a temperature at which a raw material mixture for a stick-like oil-based cosmetic is melted and injected into a mold to form a stick-like cosmetic; therefore, it is believed that the above-described orientation of a photoluminescent pigment caused by convection is reduced or inhibited, as a result of which flow marks are reduced or inhibited in the appearance of the oil-based cosmetic.

As for the appearance gloss of an oil-based cosmetic, first, it is believed to be affected by the size of a photoluminescent pigment. For example, in a photoluminescent pigment having a flake-like shape, a flat surface with a large area has the greatest contribution to the photoluminescence. Streak-like flow marks appear as, for example, a horizontal stripe pattern. In the case of a horizontal stripe pattern, a flake-like photoluminescent pigment having a large size is likely to be oriented along a convective flow such that its flat surface contributing to the photoluminescence is substantially perpendicular to the axial direction in a stick; therefore, it is believed that such a photoluminescent pigment having a large size hardly contributes to the appearance gloss of an oil-based cosmetic.

On the other hand, it is believed that a small photoluminescent pigment having an average particle size of 80 μm or less is likely to exhibit a tendency of moving in a cosmetic while randomly rotating due to convection. As a result, the flat surface of this photoluminescent pigment contributing to the photoluminescence is, as compared to that of a photoluminescent pigment having a large size, more likely to be oriented also in the direction substantially parallel to the axial direction in a stick; therefore, it is believed that the use of a small photoluminescent pigment having an average particle size of 80 μm or less can improve the surface gloss of an oil-based cosmetic in appearance.

In addition, it was revealed that the use of a large photoluminescent pigment having an average particle size of more than 80 μm (second photoluminescent pigment) in combination with a small photoluminescent pigment having an average particle size of 80 μm or less (first photoluminescent pigment) deteriorates the surface appearance of an oil-based cosmetic in some cases. This is believed to be because the above-described orientation in the large photoluminescent pigment having an average particle size of more than 80 μm inhibits random rotational movement of a smaller photoluminescent pigment. In the cosmetic of the present disclosure, when photoluminescent pigments are used in such a configuration, it is believed that the above-described problem can be solved and the appearance gloss of the oil-based cosmetic can thus be improved by controlling the mass ratio of the first photoluminescent pigment having an average particle size of 80 μm or less and the second photoluminescent pigment having an average particle size of more than 80 μm to be 55:45 to 100:0.

Further, when a photoluminescent pigment has a large size, even if a coloring material and/or thickening particles of aerosol silicic anhydride or the like are adhered to the flat surface of the photoluminescent pigment contributing to the photoluminescence, the effect thereof to reduce the photoluminescence is believed to be limited because of the large area of the flat surface. However, in the case of a photoluminescent pigment having an average particle size of lam or less, since its flat surface has a small area, adhesion of a coloring material and/or thickening particles thereto is believed to make the photoluminescence unlikely to be exerted. In the cosmetic of the present disclosure, it is believed that this problem can be solved and the appearance gloss of the oil-based cosmetic can thus be improved by controlling the content of thickening particles to be less than 4.0% by mass and, when a coloring material is used, controlling the mass ratio of the first photoluminescent pigment having an average particle size of 80 μm or less with respect to the coloring material to be 2.0 or higher.

In the oil-based cosmetic of the present disclosure, it is believed that, by adopting the above-described configuration, not only a pattern such as uneven or streak-like flow marks can be reduced or inhibited, but also an unprecedented excellent glossy texture as in the body of a brand new car or the surface of an oil-based cosmetic to which a photoluminescent pigment has been directly applied can be provided to the appearance of the oil-based cosmetic.

<<Oil-based Cosmetic>>

The oil-based cosmetic of the present disclosure can exhibit a viscosity of 50 mPa·s or higher under a temperature in at least a portion of a range of 80 to 150° C., for example, at 90° C. From the standpoints of the appearance gloss of the cosmetic, reduction or inhibition of flow marks, and the like, the viscosity may be, for example, 50 mPa·s or higher, 60 mPa·s or higher, 70 mPa·s or higher, 80 mPa·s or higher, 90 mPa·s or higher, or 100 mPa·s or higher. An upper limit value of the viscosity is not particularly limited and may be, for example, 35,000 mPa·s or lower, 30,000 mPa·s or lower, 25,000 mPa·s or lower, 20,000 mPa·s or lower, 15,000 mPa·s or lower, 10,000 mPa·s or lower, 8,000 mPa·s or lower, 5,000 mPa·s or lower, 3,000 mPa·s or lower, or 1,000 mPa·s or lower.

<Photoluminescent Pigments>

The cosmetic of the present disclosure contains a first photoluminescent pigment having an average particle size of 80 μm or less. The first photoluminescent pigment may be any of a colorless pigment, a white pigment, and a colored pigment. The colorless, white, or colored first photoluminescent pigment may be used singly, or in combination of two or more kinds thereof.

From the standpoint of the appearance gloss of the cosmetic, the first photoluminescent pigment may have an average particle size of 80 μm or less, 70 μm or less, 60 μm or less, 50 μm or less, 40 μm or less, or 30 μm or less. A lower limit value of the average particle size of the first photoluminescent pigment is not particularly limited and, from the standpoint of the appearance gloss of the cosmetic, it may be 5μm or more, 7μm or more, 10 μm or more, 13 μm or more, or 15 μm or more. The term “average particle size” used herein for a photoluminescent pigment refers to a light scattering equivalent diameter that is optically measured by a dynamic light scattering method, assuming that particles of the photoluminescent pigment have a spherical shape. According to, for example, “Saishin Funtai Bussei Zusetsu (Latest Powder Property Illustrations), Third Edition” (published on Jun. 30, 2004, written by Yutaka Kurata, publisher: NGT LLC.), the light scattering equivalent diameter is defined as the diameter of a sphere that exhibits a light scattering pattern closest to that of a particle obtained by measurement and has the same refractive index as the particle.

The amount of the first photoluminescent pigment to be incorporated is not particularly limited and, from the standpoints of the appearance gloss of the cosmetic, the aesthetics upon application of the cosmetic, and the like, the amount of the first photoluminescent pigment to be incorporated may be, for example, 2.0% by mass or more, 3.0% by mass or more, 4.0% by mass or more, or 5.0% by mass or more, with respect to a total amount of the cosmetic. An upper limit value of the amount of the first photoluminescent pigment to be incorporated is not particularly limited and may be, for example, 20% by mass or less, 15% by mass or less, 10% by mass or less, or 8.0% by mass or less.

The cosmetic of the present disclosure may also contain a second photoluminescent pigment having an average particle size of more than 80 μm within a range that does not affect the effects of the present invention and, from the standpoint of the appearance gloss of the cosmetic, it is advantageous to control the mass ratio of the first photoluminescent pigment having an average particle size of 80 μm or less and the second photoluminescent pigment having an average particle size of more than 80 pin to be in a range of 55:45 to 100:0. This mass ratio is preferably in a range of 60:40 to 100:0, more preferably in a range of 70:30 to 100:0, still more preferably in a range of 80:20 to 100:0, particularly preferably in a range of 90:10 to 100:0. The second photoluminescent pigment having a large size may deteriorate uniform appearance gloss of the cosmetic and, for example, when the cosmetic is in the form of a stick, the second photoluminescent pigment may cause the stick itself, or the lips or the like to which the cosmetic is applied, to exhibit an unnatural dotted glitter texture. Therefore, the amount of this second photoluminescent pigment to be incorporated is preferably small, and it is more preferred not to incorporate the second photoluminescent pigment.

The types of the photoluminescent pigments are not particularly limited and may be selected as appropriate, taking into consideration the aesthetics upon application of the cosmetic, the appearance gloss of the cosmetic, and the like. For example, a photoluminescent pigment generally referred to as “pearlescent pigment” can be used. Such a photoluminescent pigment may be used singly, or in combination of two or more kinds thereof.

Examples of the photoluminescent pigments include titanated mica, iron oxide-coated titanated mica, carmine-coated titanated mica, carmineiron blue-coated titanated mica, iron oxidecarmine-treated titanated mica, iron blue-treated titanated mica, iron oxideiron blue-treated titanated mica, chromium oxide-treated titanated mica, black titanium oxide-treated titanated mica, acrylic resin-coated aluminum powder, silica-coated aluminum powder, titanium oxide-coated mica, titanium oxide-coated bismuth oxychloride, titanium oxide-coated talc, colored titanium oxide-coated mica, titanium oxide-coated synthetic mica, titanium oxide-coated silica, titanium oxide-coated alumina, titanium oxide-coated glass powder, polyethylene terephthalate-polymethyl methacrylate laminated film powder, bismuth oxychloride, fish scale flake, iron oxide/titanium oxide-coated mica such as red iron oxide/titanium oxide-coated mica obtained by coating mica with iron oxide and titanium oxide, and titanium oxide in a hollow powder form in which silica is sandwiched between mica and a titanium oxide coating layer. These photoluminescent pigments typically display white or other color.

As a colorless photoluminescent pigment, any pigment known as a transparent photoluminescent pigment can be used. Examples thereof include photoluminescent pigments in which a coating film composed of a high-refractive-index material such as titanium dioxide is formed on the surface of a glass particle used as a substrate.

The term “photoluminescent pigment” used herein is intended to mean a pigment that exhibits photoluminescence without containing the below-described coloring material. The term “colored photoluminescent pigment” used herein is intended to mean a photoluminescent pigment that is not colorless and displays a color other than white. Further, the photoluminescent pigments typically take a flat-plate shape, such as a flake-like shape or a scale-like shape.

<Polymeric Oil-Phase Thickener>

The cosmetic of the present disclosure can be prepared to have the above-described viscosity using a polymeric oil-phase thickener. From the standpoint of the appearance gloss and the like of the cosmetic, it is advantageous that the polymeric oil-phase thickener be a transparent polymeric oil-phase thickener. For example, thickening particles used in the preparation of a conventional stick-like cosmetic can adhered to the flat surface of a photoluminescent pigment to cause a loss of photoluminescence as described above; however, as compared to such thickening particles, a polymeric oil-phase thickener, particularly a transparent polymeric oil-phase thickener is less likely to deteriorate the photoluminescence even if it adheres to the flat surface of a photoluminescent pigment.

The polymeric oil-phase thickener may be incorporated as appropriate such that the above-described viscosity can be exerted, and the amount thereof is not particularly limited. For example, from the standpoint of the appearance gloss, the usability, and the like of the cosmetic, the polymeric oil-phase thickener may be incorporated in an amount of 0.5% by mass or more, 1.0% by mass or more, 3.0% by mass or more, or 5.0% by mass or more, but 60% by mass or less, 50% by mass or less, 40% by mass or less, 30% by mass or less, 20% by mass or less, 17% by mass or less, 15% by mass or less, 13% by mass or less, or 10% by mass or less, with respect to a total amount of the cosmetic.

The polymeric oil-phase thickener is not particularly limited and, for example, an agent that contains a copolymer having at least one selected from an ethylene monomer unit and a styrene monomer unit can be used. For example, in a copolymer having a styrene monomer unit (styrene segment), the styrene segments in the copolymer molecules are attracted to each other in an oil phase, and this allows three-dimensional network structures to be developed between the copolymer molecules, whereby the viscosity of the oil phase can be increased.

Such a polymeric oil-phase thickener can contain specifically at least one selected from (styrene/isoprene) copolymers, (ethylene/propylene/styrene) copolymers, (styrene/butadiene) copolymers, (styrene/ethylene/butylene) copolymers, (styrene/propylene/butylene) copolymers, (styrene/butylene) copolymers, and (ethylene/propylene) copolymers. These copolymers may be hydrogenated. Thereamong, from the standpoint of the appearance gloss and the like of the cosmetic, the polymeric oil-phase thickener preferably contains at least one selected from hydrogenated (styrene/isoprene) copolymers, (ethylene/propylene/styrene) copolymers, (styrene/ethylene/butylene) copolymers, and (ethylene/propylene) copolymers.

<Thickening Particles>

For example, in a conventional stick-like oil-based cosmetic, thickening particles are typically incorporated for adjusting the viscosity of the cosmetic. However, such thickening particles may cause a loss of photoluminescence when adhered to the flat surface of the first photoluminescent pigment, making it difficult to attain good gloss in the appearance of the cosmetic.

Accordingly, the cosmetic of the present disclosure may contain thickening particles in an amount of less than 4.0% by mass, 3.5% by mass or less, 3.0% by mass or less, 2.5% by mass or less, 2.0% by mass or less, less than 2.0% by mass, 1.5% by mass or less, 1.0% by mass or less, or 0.5% by mass or less, with respect to a total amount of the cosmetic; however, from the standpoint of the appearance gloss of the cosmetic, the cosmetic preferably does not contain thickening particles.

Examples of the thickening particles include thickening inorganic particles of aerosol silicic anhydride, organo-modified clay minerals, and the like. A hydrophobization treatment may be performed on these thickening particles.

Examples of the organo-modified clay minerals include water-swellable clay minerals treated with a quaternary ammonium salt.

<Coloring Material>

The cosmetic of the present disclosure may also contain a coloring material. Particularly, when a colorless or white photoluminescent pigment is used, from the standpoint of the aesthetics and the like upon application of the cosmetic, the cosmetic preferably contains a coloring material at the same time. The term “coloring material” used herein is intended to mean a non-photoluminescent material capable of allowing the cosmetic to develop a color and, specifically, may be intended to mean a material generally referred to as “inorganic pigment”, “organic pigment”, or “dye”.

For example, from the standpoint of the appearance gloss, aesthetics, and the like of the cosmetic, the amount of the coloring material to be incorporated may be 10% by mass or less, 7.0% by mass or less, 5.0% by mass or less, 3.0% by mass or less, or 1.5% by mass or less, with respect to a total amount of the cosmetic. When the cosmetic contains a colored photoluminescent pigment, a coloring material does not have to be incorporated.

When the first photoluminescent pigment and a coloring material are used in combination, from the standpoint of the appearance gloss of the cosmetic, it is preferred to control the mass ratio of the first photoluminescent pigment with respect to the coloring material to be 2.0 or higher, 2.5 or higher, 3.0 or higher, 3.5 or higher, or 4.0 or higher. An upper limit value of this mass ratio is not particularly limited and may be, for example, 20 or lower, 15 or lower, 10 or lower, or 8.0 or lower.

The type of the coloring material is not particularly limited and may be selected as appropriate, taking into consideration the aesthetics upon application of the cosmetic, the appearance gloss of the cosmetic, and the like.

Specific examples of an inorganic pigment include: inorganic red pigments (e.g., iron oxide (red iron oxide) and iron titanate); inorganic brown pigments (e.g., γ-iron oxide); inorganic yellow pigments (e.g., yellow iron oxide and loess); inorganic black pigments (e.g., black iron oxide and low-order titanium oxide); inorganic violet pigments (e.g., manganese violet and cobalt violet); inorganic green pigments (e.g., chromium oxide, chromium hydroxide, and cobalt titanate); inorganic blue pigments (e.g., ultramarine and iron blue); inorganic white pigments (e.g., titanium dioxide and zinc oxide); and metal powders (e.g., aluminum, gold, silver, and copper).

Examples of an organic pigment include organic pigments of zirconium, barium, aluminum lake or the like, such as Red No. 2, Red No. 3, Red No. 102, Red No. 104, Red No. 105, Red No. 106, Red No. 201, Red No. 202, Red No. 203, Red No. 204, Red No. 205, Red No. 206, Red No. 207, Red No. 208, Red No. 213, Red No. 214, Red No. 215, Red No. 218, Red No. 219, Red No. 220, Red No. 221, Red No. 223, Red No. 225, Red No. 226, Red No. 227, Red No. 228, Red No. 230, Red No. 231, Red No. 232, Red No. 401, Red No. 404, Red No. 405, Red No. 501, Red No. 502, Red No. 503, Red No. 504, Red No. 505, Red No. 506, Orange No. 201, Orange No. 205, Orange No. 401, Yellow No. 4, Yellow No. 5, Yellow No. 201, Yellow No. 202, Yellow No. 203, Yellow No. 204, Yellow No. 205, Yellow No. 401, Yellow No. 402, Yellow No. 403-(1), Yellow No. 404, Yellow No. 405, Yellow No. 406, Yellow No. 407, Blue No. 1, Blue No. 404, Green No. 3, Green No. 201, Green No. 202, Green No. 204, and Violet No. 201.

Examples of a natural dye include P-carotene, cochineal dyes, red cabbage dyes, riboflavin, crocin, anthraquinone, canthaxanthin, and safflower dyes.

These coloring materials may be used singly, or in combination of two or more kinds thereof.

<Oil Component>

The cosmetic of the present disclosure may contain an oil component.

The amount of the oil component to be incorporated is not particularly limited and may be, for example, 30% by mass or more, 40% by mass or more, 50% by mass or more, or 60% by mass or more, but 95% by mass or less, 90% by mass or less, 85% by mass or less, 80% by mass or less, 75% by mass or less, or 70% by mass or less, with respect to a total amount of the cosmetic.

The oil component is not particularly limited and may be, for example, an animal oil, a vegetable oil, or a synthetic oil. As a property of the oil component, the oil component may be, for example, in a solid state, a semi-solid state, a liquid state, or a volatile state. Specific examples of the oil component include hydrocarbon oils, fatty oils, waxes, hardened oils, fatty acids, higher alcohols, silicone oils, fluorine oils, oily gelling agents, and polar oils. These oil components may be used singly, or in combination of two or more kinds thereof.

Examples of the hydrocarbon oils include liquid paraffin, heavy liquid isoparaffin, α-olefin oligomers, squalane, petroleum jelly, polyisobutylenes, hydrogenated polyisobutenes, polybutenes, hydrogenated polybutenes, polydecenes, and hydrogenated polydecenes.

Examples of the fatty oils include olive oil, castor oil, jojoba oil, mink oil, and macadamia nut oil.

Examples of the waxes include paraffin wax, ceresin wax, microcrystalline wax, polyethylene wax, montan wax, Fischer-Tropsch wax, carnauba wax, candelilla wax, Japan wax, beeswax, and spermaceti wax.

Examples of the fatty acids include stearic acid, lauric acid, myristic acid, behenic acid, isostearic acid, and oleic acid.

Examples of the higher alcohols include stearyl alcohol, cetyl alcohol, lauryl alcohol, oleyl alcohol, isostearyl alcohol, behenyl alcohol, and octyldodecanol.

Examples of the silicone oils include low-polymerization-degree dimethylpolysiloxanes, high-polymerization-degree dimethylpolysiloxanes, methylphenylpolysiloxanes, decamethylcyclopentasiloxane, octamethylcyclotetrasiloxane, polyether-modified polysiloxanes, polyoxyalkylene-alkylmethylpolysiloxane-methylpolysiloxane copolymers, alkoxy-modified polysiloxanes, crosslinked organopolysiloxanes, and fluorine-modified polysiloxanes.

Examples of the fluorine oils include perfluorodecane, perfluorooctane, and perfluoropolyethers.

Examples of the oily gelling agents include sucrose fatty acid esters, starch fatty acid esters, aluminum 12-hydroxystearate, and calcium stearate.

Examples of the polar oils include those having an IOB value of 0.10 or more, 0.15 or more, or more, 0.22 or more, or 0.24 or more. An upper limit of the IOB value is not particularly limited and may be, for example, 0.50 or less, 0.45 or less, or 0.40 or less. The “JOB value”, which is an abbreviation of “Inorganic/Organic Balance value”, is a value which represents a ratio of inorganic value with respect to organic value and serves as an index that indicates the degree of polarity of an organic compound. Specifically, the IOB value is represented by: IOB value=Inorganic value/Organic value. The “inorganic value” and the “organic value” are each set for the respective atoms and functional groups such that, for example, the “organic value” is for one carbon atom in a molecule and the “inorganic value” is 100 for one hydroxy group in a molecule, and the IOB value of an organic compound can be calculated by integrating the “inorganic values” and the “organic values” of all of the atoms and functional groups in the organic compound (see, for example, “Yuki Gainenzu—Kiso to Oyo—” (Organic Conceptual Diagrams—Fundamentals and Applications), p.11-17, published by Sankyo Shuppan Co., Ltd., 1984).

Examples of such polar oils include neopentyl glycol dicaprate, glyceryl tri-2-ethylhexanoate, pentaerythrityl tetra-2-ethylhexanoate, caprylic/capric triglyceride, diethylhexyl sebacate, octyldodecanol, glyceryl diisostearate, diglyceryl triisostearate, diisostearyl malate, trimethylolpropane tri-2-ethylhexanoate, oxystearyl oxystearate, pentaerythrityl tetra(ethylhexanoate/benzoate), trioctanoin, pentaerythrityl tetraoctanoate, dipentaerythrityl hexahydroxystearate, castor oil, diisopropyl sebacate, pentaerythritol tetraoctanoate, and trimethylolpropane triisostearate.

These oil components can each be used as a base of the oil-based cosmetic. When the base itself has excellent transparency, for example, a photoluminescent pigment existing on the inner side than the surface of the oil-based cosmetic can also contribute to the reflection of light coming from the outside; therefore, the appearance gloss of the oil-based cosmetic can be further improved. From the standpoint of improving the transparency of the base itself and thereby further improving the appearance gloss of the oil-based cosmetic, among the above-exemplified oil components, at least one selected from hydrogenated polyisobutene, microcrystalline wax, polyethylene wax, octyldodecanol, diisostearyl malate, and neopentyl glycol dicaprate is preferably used.

On the other hand, for example, diglyceryl triisostearate, diglyceryl tetraisostearate, pentaerythrityl tetraoctanoate, dipentaerythrityl tetraisostearate, and dipentaerythrityl hexahydroxystearate present a risk of deteriorating the transparency of the base itself; therefore, although the cosmetic may contain these oil components in a range of 2.0% by mass or less, less than 2.0% by mass, 1.5% by mass or less, 1.0% by mass or less, less than 1.0% by mass, 0.5% by mass or less, or less than 0.5% by mass, with respect to a total amount of the cosmetic, it is preferred not to incorporate these oil components into the cosmetic.

<Optional Components>

In the oil-based cosmetic of the present disclosure, in addition to the above-described components, various components can be incorporated as appropriate within a range that does not affect the effects of the present invention. Examples of the various components include a moisturizer, a preservative, an anti-foaming agent, an antioxidant, an ultraviolet absorber, a polymer, a surfactant, a polymeric oil-phase thickener, and a thickener other than thickening particles (hereinafter, may be referred to as “second thickener”), a drug, an alcohol, an antibacterial agent, a solvent, and a fragrance. These optional components may be used singly, or in combination of two or more kinds thereof.

The ultraviolet absorber is not particularly limited, and examples thereof include organic ultraviolet absorbers, such as ethylhexyl methoxycinnamate, octocrylene, polysilicone-15, t-butyl methoxydibenzoylmethane, ethylhexyl triazone, bis-ethylhexyloxyphenol methoxyphenyl triazine, diethylamino hydroxybenzoyl hexyl benzoate, oxybenzone-3, methylene bis-benzotriazolyltetramethylbutylphenol, homosalate, and ethylhexyl salicylate. These ultraviolet absorbers may be used singly, or in combination of two or more thereof. It is noted here that these organic ultraviolet absorbers may be regarded as oil components.

<<Method of Producing Oil-based Cosmetic>>

A method of producing the oil-based cosmetic of the present disclosure is not particularly limited, and any known production method can be employed. For example, a stick-like oil-based cosmetic can be obtained in the following manner.

A raw material mixture for a stick-like oil-based cosmetic, in which mixture contains the above-described first photoluminescent pigment having an average particle size of 80 μm or less, polymeric oil-phase thickener and, as required, optional components such as a coloring material, is kneaded with heating in a temperature range of 80 to 150° C. to prepare an oil-based composition such that this composition has a viscosity of 50 mPa·s or higher under a temperature in at least a portion of a range of 80 to 150° C., for example, at 90° C. In this process, such a specific viscosity can be typically attained by adjusting the polymeric oil-phase thickener and the amount thereof. The polymeric oil-phase thickener may be used after being mixed with and dissolved in the above-described oil component.

The oil-based composition which has been heat-melted such that it exhibits the prescribed viscosity as described above is injected into a stick-shaped mold. The oil-based composition is cooled to room temperature (e.g., 0 to 30° C.) and subsequently removed from the mold, whereby a stick-like oil-based cosmetic can be obtained. As the mold, from the standpoint of the surface gloss and the like of the resulting stick in appearance, it is preferred to employ a mold made of a silicone resin, a metal, or a metal alloy.

<<Form and Use of Oil-based Cosmetic>>

The oil-based cosmetic of the present disclosure may be in the form of a highly viscous liquid having a viscosity of, for example, 10,000 mPa·s or higher (e.g., a cream form), or in the form of a solid; however, the above-described effects of the present invention can be exerted more prominently when the oil-based cosmetic is in the form of a solid. Examples of a solid-form oil-based cosmetic include, but not particularly limited to: stick-type and inner tray-type cosmetics. Thereamong, the oil-based cosmetic of the present disclosure can be preferably used as a stick-type cosmetic. The term “solid-form oil-based cosmetic” used herein may be intended to mean, for example, an oil-based cosmetic exhibiting a solid form at 30° C.

The use of the oil-based cosmetic of the present disclosure is not particularly limited, and examples thereof include: lip cosmetics, such as lipsticks, lip glosses, and lip creams; make-up cosmetics, such as foundations and eyeshadows; and hair cosmetics, such as hair sticks and pomades. Thereamong, the cosmetic of the present disclosure is advantageously used as a lipstick that is likely to give a visual sense of luxury associated with the surface gloss in appearance.

### EXAMPLES

The present invention will now be described in more detail by way of Examples; however, the present invention is not limited thereto. Hereinafter, unless otherwise specified, amounts are indicated in % by mass.

<<Examples 1 to 7 and Comparative Examples 1 to 7>>

Oil-based cosmetics obtained by the below-described production method in accordance with the respective formulations shown in Table 1 were each evaluated as described below, and the results thereof are shown in Tables 1 to 3. It is noted here that the oil-based cosmetics of Examples 1 to 6 and Comparative Examples 1 to 6 are examples of a stick-type oil-based cosmetic, while the oil-based cosmetics of Example 7 and Comparative Example 7 are examples of an inner tray-type oil-based cosmetic.

<Evaluation Methods>

(Evaluation of Viscosity)

The viscosity of a cosmetic was evaluated using a B-type viscometer (TVB-type viscometer TVB-10, manufactured by Toki Sangyo Co., Ltd.) under the following conditions: 90° C., rotor number=M2 or H7, and 20 rpm. As the rotor, H7 was used in Example 3, while M2 was used in other Examples and Comparative Examples. The “viscosity of a cosmetic” is intended to mean the viscosity of the below-described oil-based composition prior to being injected into a mold.

(Evaluation of Flow Marks)

Flow marks of each of the oil-based cosmetics prepared by the below-described method were visually observed and evaluated based on the following criteria. The evaluations of A to C can be deemed to be acceptable, while the evaluation of D can be deemed to be unacceptable.

A: Uneven or streak-like flow marks were not observed.

B: Uneven or streak-like flow marks were very slightly observed.

C: Uneven or streak-like flow marks were slightly observed.

D: Uneven or streak-like flow marks were clearly observed.

(Evaluation of Surface Gloss)

The surface gloss in appearance of each of the oil-based cosmetics prepared by the below-described method were visually observed and evaluated based on the following criteria. The evaluations of A to C can be deemed to be acceptable, while the evaluation of D can be deemed to be unacceptable.

A: The oil-based cosmetic had excellent surface gloss like the body of a brand new car.

B: A slight dullness was observed as compared to the surface gloss of the evaluation A.

C: A slight dullness was observed as compared to the surface gloss of the evaluation B.

D: A dullness was observed as compared to the surface gloss of the evaluation C.

(Evaluation of Unevenness in Finish after Application)

The oil-based cosmetics prepared by the below-described method were each applied to the inside of the arm, and the appearance thereof was visually observed and evaluated based on the following criteria.

A: An unevenness in finish was not observed at all.

B: An unevenness in finish was slightly observed.

C: An unevenness in finish was clearly observed.

<Method of Producing Stick-Type Oil-based Cosmetics>

### (Examples 1 to 6 and Comparative Examples 1 to 6)

Stick-type oil-based cosmetics of Examples 1 to 6 and Comparative Examples 1 to 6 were each produced by the following method using the respective formulations shown in Table 1.

All of the materials were mixed, heat-melted at 100° C., and subsequently dispersed using a homodisper to prepare an oil-based composition.

This oil-based composition heat-melted at 90° C. was injected into a stick-shaped mold made of a metal or a silicone resin. The mold was cooled to room temperature and, after confirming that the composition was sufficiently solidified, the resulting cosmetic was removed from the mold, whereby a stick-type oil-based cosmetic was obtained.

TABLE 1

| | | Comparative | Example | Example | Example |
|---|---|---|---|---|---|
| Components | Component name or product name | Example 1 | 1 | 2 | 3 |
| Oil component (non-polar oil) | Mixture of polyethylene wax and microcrystalline wax | 12.00 | 12.00 | 12.00 | 12.00 |
| First photoluminescent pigment | Iron oxide·titanium oxide-coated mica having an | 5.00 | 5.00 | 5.00 | 5.00 |
| | average particle size of about 20 μm | | | | |
| Second photoluminescent pigment | Titanium oxide-coated glass having an average particle | — | 1 | — | — |
| | size of about 100 μm | | | | |
| Polymeric oil-phase thickener | VERSAGEL (trademark) ME2000¹⁾ | 10.00 | 20.00 | 30.00 | 50.00 |
| (transparent) | | | | | |
| Oil component (non-polar oil) | Hydrogenated polyisobutene (viscosity = 20 mPa · s) | 39.93 | 29.93 | 19.93 | |
| | Hydrogenated polyisobutene (viscosity = 15,000 | 10.00 | 10.00 | 10.00 | 9.93 |
| | mPa · s) | | | | |
| Higher alcohol | Octyldodecanol | 10.00 | 10.00 | 10.00 | 10.00 |
| Oil component (polar oil) | Neopentyl glycol dicaprate | 3.00 | 3.00 | 3.00 | 3.00 |
| Ultraviolet absorber (polar oil) | Ethylhexyl methoxycinnamate | 2.00 | 2.00 | 2.00 | 2.00 |
| Oil component (polar oil) | Diisostearyl malate | 5.00 | 5.00 | 5.00 | 5.00 |
| Anti-foaming agent | Simethicone | 0.02 | 0.02 | 0.02 | 0.02 |
| Antioxidant | Tocopherol | 0.05 | 0.05 | 0.05 | 0.05 |
| Thickening particles | Hydrophobized aerosol silicic anhydride | | | - | |
| Coloring material | Red coloring material paste having a pigment content | 3.00 | 3.00 | 3.00 | 3.00 |
| | of 40% | | | | |
| Total | | 100 | 100 | 100 | 100 |
| Mass ratio of first photoluminescent pigment and second photoluminescent pigment | | 100:0 | 100:0 | 100:0 | 100:0 |
| Mass ratio of first photoluminescent pigment with respect to coloring material | | 4.2 | 4.2 | 4.2 | 4.2 |
| Evaluation | Viscosity at 90° C. (mPa · s) | 34 | 82 | 650 | 7,200 |
| | Flow marks | D | A | A | A |
| | Surface gloss | A | A | A | A |
| | Unevenness in finish after application | A | A | A | A |
| | | Example | Comparative | Comparative | Comparative |
| Components | Component name or product name | 4 | Example 2 | Example 3 | Example 4 |
| Oil component (non-polar oil) | Mixture of polyethylene wax and microcrystalline wax | 12.00 | 12.00 | 12.00 | 12.00 |
| First photoluminescent pigment | Iron oxide·titanium oxide-coated mica having an | 5.00 | 5.00 | 2.00 | 5.00 |
| | average particle size of about 20 μm | | | | |
| Second photoluminescent pigment | Titanium oxide-coated glass having an average particle | — | 5.00 | — | — |
| | size of about 100 μm | | | | |
| Polymeric oil-phase thickener | VERSAGEL (trademark) ME2000¹⁾ | 5.00 | 20.00 | 20.00 | |
| (transparent) | | | | | |
| Oil component (non-polar oil) | Hydrogenated polyisobutene (viscosity = 20 mPa · s) | 42.93 | 24.93 | 32.93 | 47.93 |
| | Hydrogenated polyisobutene (viscosity = 15,000 | 10.00 | 10.00 | 10.00 | 10.00 |
| | mPa · s) | | | | |
| Higher alcohol | Octyldodecanol | 10.00 | 10.00 | 10.00 | 10.00 |
| Oil component (polar oil) | Neopentyl glycol dicaprate | 3.00 | 3.00 | 3.00 | 3.00 |
| Ultraviolet absorber (polar oil) | Ethylhexyl methoxycinnamate | 2.00 | 2.00 | 2.00 | 2.00 |
| Oil component (polar oil) | Diisostearyl malate | 5.00 | 5.00 | 5.00 | 5.00 |
| Anti-foaming agent | Simethicone | 0.02 | 0.02 | 0.02 | 0.02 |
| Antioxidant | Tocopherol | 0.05 | 0.05 | 0.05 | 0.05 |
| Thickening particles | Hydrophobized aerosol silicic anhydride | 2.00 | | | 2.00 |
| Coloring material | Red coloring material paste having a pigment content | 3.00 | 3.00 | 3.00 | 3.00 |
| | of 40% | | | | |
| Total | | 100 | 100 | 100 | 100 |
| Mass ratio of first photoluminescent pigment and second photoluminescent pigment | | 100:0 | 50:50 | 100:0 | 100:0 |
| Mass ratio of first photoluminescent pigment with respect to coloring material | | 4.2 | 4.2 | 1.6 | 4.2 |
| Evaluation | Viscosity at 90° C. (mPa · s) | 70 | 200 | 84 | 180 |
| | Flow marks | B | A | A | B |
| | Surface gloss | B | D | D | D |
| | Unevenness in finish after application | A | A | A | A |

¹⁾Mixture of a (styrene/ethylene/propylene) copolymer, a (styrene/ethylene/butylene) copolymer, and a hydrogenated polyisobutene

TABLE 2

| | | Comparative | Comparative | | |
|---|---|---|---|---|---|
| Components | Component name or product name | Example 5 | Example 6 | Example 5 | Example 6 |
| Oil component (non-polar oil) | Mixture of polyethylene wax and | 12.00 | 12.00 | 12.00 | 12.00 |
| | microcrystalline wax | | | | |
| First photoluminescent pigment | Iron oxide•titanium oxide-coated mica | 5.00 | — | 5.00 | 5.00 |
| | having an average particle size of | | | | |
| | about 20 μm | | | | |
| Second photoluminescent pigment | Titanium oxide-coated glass having an | — | 5.00 | — | — |
| | average particle size of about 100 μm | | | | |
| Polymeric oil-phase thickener | VERSAGEL (trademark) ME2000¹⁾ | 10.00 | — | — | — |
| (transparent) | CREAGEL (trademark) Crystal AF²⁾ | — | — | 30.00 | — |
| | PIONIER GEL 12PAO³⁾ | — | — | — | 30.00 |
| Oil component (non-polar oil) | Hydrogenated polyisobutene | 35.93 | 49.93 | 19.93 | 19.93 |
| | (viscosity = 20 mPa · s) | | | | |
| | Hydrogenated polyisobutene | 10.00 | 10.00 | 10.00 | 10.00 |
| | (viscosity = 15,000 mPa · s) | | | | |
| Higher alcohol | Octyldodecanol | 10.00 | 10.00 | 10.00 | 10.00 |
| Oil component (polar oil) | Neopentyl glycol dicaprate | 3.00 | 3.00 | 3.00 | 3.00 |
| Ultraviolet absorber (polar oil) | Ethylhexyl methoxycinnamate | 2.00 | 2.00 | 2.00 | 2.00 |
| Oil component (polar oil) | Diisostearyl malate | 5.00 | 5.00 | 5.00 | 5.00 |
| Anti-foaming agent | Simethicone | 0.02 | 0.02 | 0.02 | 0.02 |
| Antioxidant | Tocopherol | 0.05 | 0.05 | 0.05 | 0.05 |
| Thickening particles | Hydrophobized aerosol silicic anhydride | 4.00 | — | — | — |
| Coloring material | Red coloring material paste having | 3.00 | 3.00 | 3.00 | 3.00 |
| | a pigment content of 40% | | | | |
| Total | | 100 | 100 | 100 | 100 |
| Mass ratio of first photoluminescent pigment | | 100:0 | 0:100 | 100:0 | 100:0 |
| and second photoluminescent pigment | | | | | |
| Mass ratio of first photoluminescent pigment | | 4.2 | 0 | 4.2 | 4.2 |
| with respect to coloring material | | | | | |
| Evaluation | Viscosity at 90° C. (mPa · s) | 1,080 | 0 | 1,448 | 657 |
| | Flow marks | A | D | A | A |
| | Surface gloss | D | D | A | A |
| | Unevenness in finish after application | A | A | A | A |

¹⁾Mixture of a (styrene/ethylene/propylene) copolymer, a (styrene/ethylene/butylene) copolymer, and a hydrogenated polyisobutene
²⁾Mixture of a (ethylene/propylene) copolymer and a hydrogenated polydecene
³⁾Mixture of a hydrogenated (styrene/isoprene) copolymer and a hydrogenated polydecene

<Results>

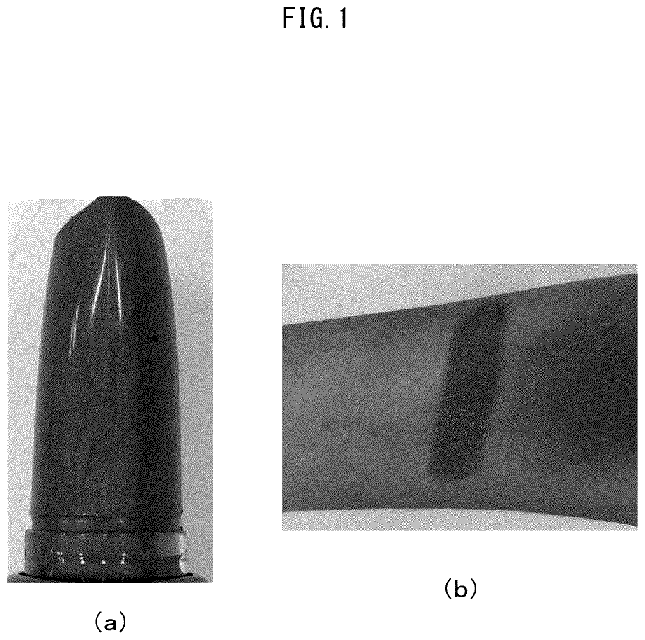

As apparent from the results of Examples 1 to 4 shown in Table 1, it was confirmed that the cosmetics were increased in viscosity under a high temperature even with a formulation containing a small amount of thickening particles, and that flow marks were reduced or inhibited when the cosmetics had a viscosity of 50 mPa·s or higher. In addition, as also seen from the results of Comparative Example 1 and FIG. 1, the unevenness in finish after application was not affected even when the cosmetic had flow marks. In other words, it was found that, even if various documents clearly state that the unevenness in finish after application is improved, the results thereof do not necessarily mean that flow marks are improved.

Further, as seen from comparisons between Examples 1 to 4 and Comparative Examples 1 to 4, it was confirmed that the surface gloss of a cosmetic in appearance is also improved by using a relatively small first photoluminescent pigment having an average particle size of 80 μm or less, and controlling the mass ratio of the first photoluminescent pigment and a larger second photoluminescent pigment, the mass ratio of the first photoluminescent pigment and a coloring material, and the content of thickening particles to be in specific ranges.

From the results of Comparative Example 5 shown in Table 2, it was confirmed that the surface gloss of a cosmetic in appearance is deteriorated when the amount of incorporated thickening particles is 4.0% by mass or more.

From the results of Comparative Example 6 shown in Table 2, it was also found that not only the surface gloss of a cosmetic in appearance is deteriorated but also flow marks are more likely to appear when a thickener is not used and the ratio of the second photoluminescent pigment having an average particle size of more than 80 μm is increased.

From the results of Examples 1 to 4 shown in Table 1 and the results of Examples 5 and 6 shown in Table 2, it was confirmed that various polymeric oil-phase thickeners can exert an effect of reducing or inhibiting flow marks and an effect of improving the surface gloss of a cosmetic in appearance.

<Method of Producing Inner Tray-Type Oil-based Cosmetics>

### (Example 7 and Comparative Example 7)

Inner tray-type oil-based cosmetics of Example 7 and Comparative Example 7 were each produced by the following method using the respective formulations shown in Table 3.

All of the materials were mixed, heat-melted at 100° C., and subsequently dispersed using a homodisper to prepare an oil-based composition. This oil-based composition heat-melted at 90° C. was injected into an inner tray-shaped metal container and subsequently cooled to room temperature, whereby an inner tray-type oil-based cosmetic was obtained.

TABLE 3

| | | Comparative | |
|---|---|---|---|
| Components | Component name or product name | Example 7 | Example 7 |
| Second thickener | Dextrin palmitate | 10.00 | 10.00 |
| First photoluminescent pigment | Iron oxide•titanium oxide-coated | 5.00 | 5.00 |
| | mica having an average particle | | |
| | size of about 20 μm | | |
| Polymeric oil-phase thickener | VERSAGEL (trademark) ME2000¹⁾ | — | 30.00 |
| (transparent) | | | |
| Oil component (non-polar oil) | Hydrogenated polyisobutene | 61.93 | 31.93 |
| | (viscosity = 15,000 mPa · s) | | |
| Oil component (polar oil) | Diisostearyl malate | 20.00 | 20.00 |
| Anti-foaming agent | Simethicone | 0.02 | 0.02 |
| Antioxidant | Tocopherol | 0.05 | 0.05 |
| Coloring material | Red coloring material paste | 3.00 | 3.00 |
| | having a pigment content of 40% | | |
| Total | | 100 | 100 |
| Mass ratio of first photoluminescent pigment | | 100:0 | 100:0 |
| and second photoluminescent pigment | | | |
| Mass ratio of first photoluminescent pigment | | 4.2 | 4.2 |
| with respect to coloring material | | | |
| Evaluation | Viscosity at 90° C. (mPa · s) | 643 | 572 |
| | Flow marks | B | A |
| | Surface gloss | D | A |
| | Unevenness in finish after application | A | A |

¹⁾Mixture of a (styrene/ethylene/propylene) copolymer, a (styrene/ethylene/butylene) copolymer, and a hydrogenated polyisobutene

<Results>

As apparent from the results of Example 7 and Comparative Example 7, it was confirmed that the effects according to the present invention of reducing or inhibiting flow marks and improving the surface gloss of a cosmetic in appearance are not limited to stick-type cosmetics, and can be exerted in the same manner even in cosmetics of an inner tray-type or the like.

1. An oil-based cosmetic, containing: a first photoluminescent pigment having an average particle size of 80 μm or less; and a polymeric oil-phase thickener,
wherein
the mass ratio of the first photoluminescent pigment and a second photoluminescent pigment having an average particle size of more than 80 μm is 55:45 to 100:0,
when the oil-based cosmetic contains a coloring material, the mass ratio of the first photoluminescent pigment with respect to the coloring material is 2.0 or higher,
the content of thickening particles is less than 4.0% by mass, and
the oil-based cosmetic has a viscosity of 50 mPa·s or higher under a temperature in at least a portion of a range of 80 to 150° C.

2. The cosmetic according to claim 1, wherein the content of the first photoluminescent pigment is 2.0% by mass or more.

3. The cosmetic according to claim 1, wherein the polymeric oil-phase thickener contains a copolymer having at least one selected from an ethylene monomer unit and a styrene monomer unit.

4. The cosmetic according to claim 1, further containing a hydrocarbon oil.

5. The cosmetic according to claim 1, containing a coloring material,
wherein the first photoluminescent pigment is a colorless or white photoluminescent pigment.

6. The cosmetic according to claim 1, which is used as a lipstick.

7. A method of producing the cosmetic according to claim 1, the method comprising:
melting a raw material mixture, which contains the first photoluminescent pigment and the polymeric oil-phase thickener, at a temperature of 80 to 150° C. at which the mixture has a viscosity of 50 mPa·s or higher; and
injecting the thus molten mixture into a mold.
